(19) United States
(12) Patent Application Publication
(10) Pub. No.: US 2004/0162482 A1
(43) Pub. Date: Aug. 19, 2004

(54) MRT apparatus, method and computer program product for speed-resolved flow measurement

(76) Inventors: Stefan Assmann, Erlangen (DE); Oliver Schreck, Bamberg (DE)

Correspondence Address: SCHIFF HARDIN & WAITE, Patent Department, 6600 Sears Tower, 233 South Wacker Drive, Chicago, IL, 60606, US

(21) Appl. No.: 10/725,622

(22) Filed: Dec. 2, 2003

(30) Foreign Application Priority Data
Feb. 12, 2002 (DE) 10256208.3

(51) Int. Cl. A61B 5/055

(52) U.S. Cl. 600/419; 382/128; 324/306

(57) ABSTRACT

In a process for speed-resolved flow measurement during a movement cycle in magnetic resonance tomography, an overview image (localizer) of a selected region of a living subject is acquired using an MRT-device, the overview image (localizer) is displayed on a screen, a quasi-simultaneous acquisition of an anatomical image series of the selected region and of a speed-resolved image series of a region identified within the selected region during the movement cycle, are performed, the two image series are displayed on the screen, and when displaying the image series, each speed-resolved image of the speed-resolved image series is integrated in the time-corresponding anatomical image of the anatomical image series.

### BACKGROUND OF THE INVENTION

1. Field of the Invention

The present invention generally involves magnetic resonance tomography, or MRT, as applied in medicine for examining patients. The present invention relates especially to a process for improving flow measurements as they are performed in magnetic resonance tomography, for example, to show vascular systems that have blood flowing through them.

2. Description of the Prior Art

MRT is based on the physical phenomenon of nuclear magnetic resonance and has been used successfully as an imaging modality for over 15 years in medicine and biophysics. In this examination method, an object is exposed to a strong, constant magnetic field. In the process, the nuclear spins of the atoms in the object, which were previously randomly oriented, become aligned. Radio-frequency energy then can excite these “aligned” nuclear spins into a certain oscillation. This oscillation generates the actual MRT measurement signal, which is acquired using suitable receiver coils. By using non-homogenous magnetic fields, generated by gradient coils, signals from the measurement object can be spatially coded in all three spatial directions, which, in general, are called “spatial coding”.

The recording of data in MRT is done in k-space (frequency domain). The MRT-image in the image domain is linked using Fourier transformation to the MRT-data in k-space. The spatial coding of the object that spans k-space is done using the aforementioned gradients in all three spatial directions. In the process, a distinction is made between the slice or layer (specifies the recorded slice in the object, usually the z-axis), the frequency coding (specifies a direction in the slice, usually the x-axis) and the phase coding (determines the second dimension within the slice, usually the y-axis). Furthermore, by phase coding along the z-axis, the selected slice can be subdivided into sub-slices.

Thus, initially a slice is selectively excited, for example, in the z-direction, and phase-coding is possibly performed in the z-direction. The coding of the spatial information in the slice is done though a combined phase and frequency coding using the two aforementioned orthogonal gradient fields, which, in the example of a slice excited in the z-direction, are generated by the aforementioned gradient fields in the x- and y-directions.

A possible form for recording the data in an MRT measurement is shown in FIGS. 4A and 4B. The sequence applied is a spin-echo sequence. In this sequence, the magnetization of the spins is made in the x-y plane by a 90° excitation pulse. In the course of time (½ T_{E}; T_{E} is the echo time), a dephasing of the magnetization portions, which together form the transverse magnetization in the x-y plane M_{xy}, occurs. After a certain time (e.g. ½ T_{E}), a 180° pulse is emitted in the x-y plane such that the dephased magnetization components are reflected without changing the precession direction and precession speed of the individual magnetization portions. After an additional time period ½ T_{E}, the magnetization components point in the same direction again, i.e. a regeneration of the transverse magnetization results (called “rephasing”). The complete regeneration of the transverse magnetization is called spin-echo.

In order to measure a corporate slice of the object to be examined, the imaging sequence is repeated N-times for different values of the phase coding gradients, e.g. G^{y}. The time interval of the respective excitation producing HF-pulses is called the repetition time TR. The magnetic resonance signal (spin-echo signal) is also scanned, digitized, and stored, in the presence of the read-out gradients G^{x}, N-times at equivalent time intervals Δt in each sequence pass by the Δt-clocked ADC (Analog Digital Converter). In this way, according to FIG. 4B, a numerical matrix that is created line-by-line (matrix in the k-space or k-matrix) with N×N data points. From this dataset, using a Fourier transformation, a MR-image of the slice in question can be directly reconstructed with a resolution of N×N pixels (a symmetrical matrix with N×N points is only one example, asymmetric matrices also can be generated).

For speed-indicating flow measurements in magnetic resonance tomography, either the progression of the average speed of the flowing medium in a certain vessel can be determined during a movement cycle (breathing, heart movement) or the speed distribution in the cross-section of the vessel region that is of interest and in which a fluid is flowing through can be determined at a defined point in time of the movement. Of great interest, for example, is the speed progression (curve) of the blood in the aorta during a cardiac cycle (from systole to systole).

At present, for measurements of this type, during the movement, i.e. within a cycle to be measured, quasi-simultaneous two-part datasets are acquired: an anatomical image series and a speed-coded image series. Usually, the acquisition frequency for the two series is approximately 20 images per cycle. The simultaneity of the image acquisition is realized by alternatingly acquiring one image of the one series and then one image of the other series and during the acquisition of the speed-coded series, a constant gradient is established in the flow direction, which is adapted to the various sequence parameters (repetition time, flip angle, etc.) and the flow speed in the vessel involved, in order to achieve an optimal speed resolution. Typically, the acquired slice of both series is oriented perpendicularly to the vessels to be depicted. The additional (phase-coding) gradient in the flow direction is therefore necessary in order to be able to assign a defined speed to each voxel of the flowing medium because of the speed-dependent dephasing and thus the intensity of the resonance signal of the nuclear spin contained therein.

Conventionally, both series have been displayed using post-processing software and evaluated primarily after the end of the examination on the patient. Accordingly, no visualization of the results of the flow measurement takes place directly after the data acquisition. The anatomical and speed-coded image series can at present only be shown separately after post-processing.

### SUMMARY OF THE INVENTION

An object of the present invention is to provide a process to realize an immediate processing (on-line) and improved preparation of the measurement results in flow measurements in magnetic resonance tomography.

According to the invention, the above object is achieved by a process for speed-resolved flow measurement during a movement cycle in magnetic resonance tomography, including: acquiring an overview image (localizer) of a selected region of a living subject to be examined using an MRT-device, displaying the overview image (localizer) on a screen, performing a quasi-simultaneous measurement of an anatomical image series of the selected region and a speed-resolved image series of a region identified within the selected region during the movement cycle, and displaying the two image series on the screen, when displaying the image series, each speed-resolved image of the speed-resolved image series is integrated in the time-corresponding anatomical image of the anatomical image series.

Preferably, as early as during or directly after the measurement, an automatic segmenting of the identified region is done via the speed-resolved image series. In this manner the contour of the region to be measured, changing under certain circumstances, can be followed. Common segmenting algorithms are known.

In order to make it easier for the user to interpret and/or diagnose, based on the image series shown, a color coding of the speed-resolved image series should be done.

A color coding of this type can be realized based on the state of the art for ultrasound image reproduction.

The processing of the measurement data according to the invention, even during or immediately after the actual measurement, makes it possible for the measurement result to be displayed immediately after the measurement, in the form of a suitably arranged image series or in the form of a film on a user-interface on the screen.

According to the invention, the tissue region to be measured is manually identified by the user. In this way, several vessel regions can also be simultaneously identified in the overview image (localizer) and then simultaneously measured in a speed-resolved manner.

According to the invention, the speed-resolving measurement of vessels is dependent on a movement cycle of the object to be examined. A movement cycle of this type can be the time period of breathing, heart movement, or other movement forms. In the process, a good resolution of the image series occurs at approximately 20 images per cycle.

The above object also is achieved in accordance with the present invention by a magnetic resonance tomography device operable to perform the above-described method.

The above object also is achieved in accordance with the present invention by a computer software product which implements a method as described above and that runs on a computer device associated with a magnetic resonance tomography device.

### DESCRIPTION OF THE DRAWINGS

FIG. 1 illustrates schematically illustrates a magnetic resonance tomography device operable in accordance with the invention.

FIG. 2a illustrates a localizer in the form of a transverse cross-section of the aorta in the mediastinum.

FIG. 2b illustrates the localizer in which the region for the speed analysis (cross-section of the aorta) is characterized as a circular ROI (region of interest).

FIG. 2c illustrates the combination of an anatomical image with the corresponding speed-coded image in the ROI.

FIG. 2d illustrates the enlargement of the speed-coded image in the ROI.

FIG. 3A illustrates, in a sectional view, an excitation layer perpendicular to a vessel that as blood flowing through it.

FIG. 3B schematically illustrates the saturation progression of the longitudinal magnetization of the excitation layer.

FIG. 3C schematically illustrates the saturation progression of the magnetization of the blood flowing into the excitation layer.

FIG. 4A schematically illustrates the time progression of the gradient pulse flow functions of a spin-echo sequence.

FIG. 4B schematically illustrates the time scanning of the k-matrix in a spin-echo sequence according to FIG. 4A.

### DESCRIPTION OF THE PREFERRED EMBODIMENTS

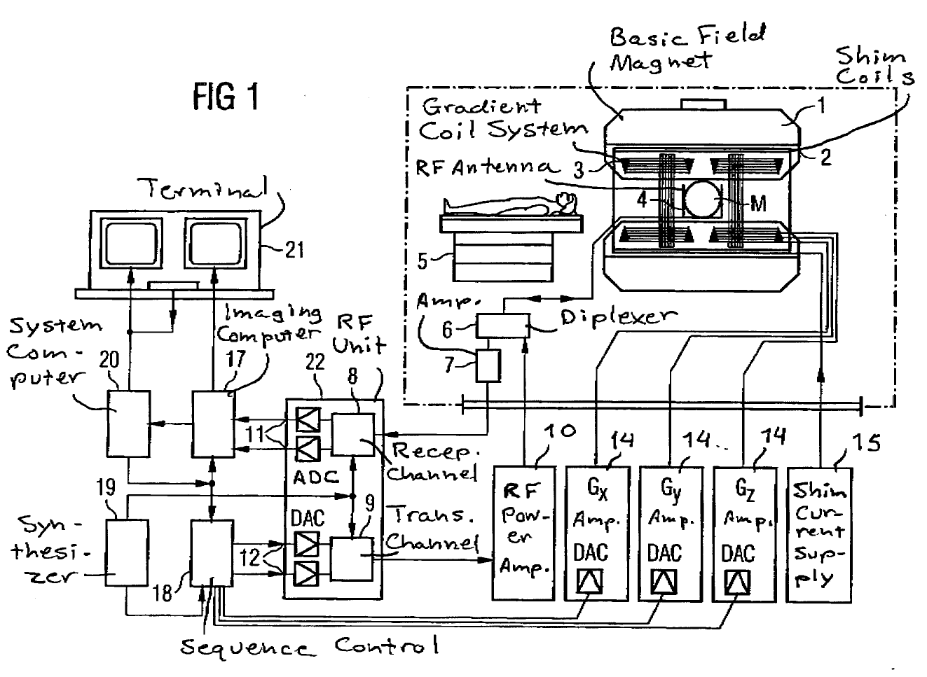

FIG. 1 is a schematic block diagram of a magnetic resonance tomography device with which optimized flow measurements according to the present invention are possible. The components of the magnetic resonance tomography device correspond to those of a conventional tomography device, with operational differences as described below. A basic field magnet 1 generates a strong magnetic field, which is constant in time, for the polarization or alignment of the nuclear spins in the examination region of an object, such as, for example, a part of a human body to be examined. The high homogeneity of the basic magnetic field required for the magnetic resonance measurement is defined in a spherical measurement volume M, into which the parts of the human body to be examined are brought. In order to satisfy the homogeneity requirements and especially for the elimination of time-invariant influences, shim-plates made of ferromagnetic material are mounted at suitable positions. Time-variable influences are eliminated by shim coils 2, which are controlled by a shim-current supply 15.

In the basic magnetic field 1, a cylinder-shaped gradient coil system 3 is used, which consists of three windings. Each winding is supplied with current by an amplifier 14 in order to generate a linear gradient field in the respective directions of the Cartesian coordinate system. The first winding of the gradient field system 3 generates in a gradient Gₓ in the x-direction, the second winding generates a gradient G_{y} in the y-direction, and the third winding generates a gradient G_{z} in the z-direction. Each amplifier 14 contains a digital-analog converter, which is controlled by a sequence control 18 for the generation of gradient pulses at proper times.

Within the gradient field system 3, a radio-frequency antenna 4 is located which converts the radio-frequency pulses emitted by a radio-frequency power amplifier 30 into a magnetic alternating field in order to excite the nuclei and align the nuclear spins of the object to be examined or the region of the object to be examined. From the radio-frequency antenna 4, the alternating field emerging from the preceding nuclear spins, i.e. usually the nuclear spin echo signals brought about by a pulse sequence from one or more high-frequency pulses and one or more gradient pulses, is converted into a voltage that is supplied via an amplifier 7 to a radio-frequency receiver channel 8 of a radio-frequency system 22. The radio-frequency system 22 contains, furthermore, a transmission channel 9, in which the radio-frequency pulses are generated for the excitation of the nuclear magnetic resonance. In the process, the respective radio-frequency pulses based on a pulse sequence specified by the system computer 20 in the sequence control 18 are represented digitally as complex numbers. This numerical sequence is supplied as real and imaginary parts via responsive inputs 12 to a digital-analog converter in the high-frequency system 22 and from there to a transmission channel 9. In the transmission channel 9, the pulse sequences are modulated with a radio-frequency carrier signal, having a base frequency corresponding to the resonance frequency of the nuclear spins in the measurement volume.

The conversion from transmitting to receiving operation is done via a diplexer 6. The radio-frequency antenna 4 emits the radio-frequency pulse to excite the nuclear spin into the measurement volume M and scans the resultant echo signals. The correspondingly obtained magnetic resonance signals are demodulated in the receiving channel 8 of the radio-frequency system 22 in a phase-sensitive manner, and are converted via respective analog-digital converter into a real part and an imaginary part of the measurement signal. Using an imaging computer 17, an image is reconstructed from the measurement data obtained in this way. The administration of the measurement data, the image data and the control programs is done via the system computer 20. Based on a specification with control programs, the sequence control 18 controls the generation of the desired pulse sequences and the corresponding scanning of k-space. In particular, the sequence control 18 controls the switching of the gradients at appropriate times, the transmission of the radio-frequency pulses with a defined phase and amplitude, and the reception of the magnetic resonance signals. The time basis for the radio-frequency system 22 and the sequence control 18 is furnished by a synthesizer 19. The selection of appropriate control programs for generating an MR image and the display of the generated nuclear spin image is done via a terminal (console) 21, which contains a keyboard and one or more screens.

The MRT-device described should be able to be configured for flow measurements according to the invention via a so-called “exam card”. The exam card is a virtual user interface which is presented to the user on the screen of the terminal 21. With it, for example, the speed-coded gradient can be adjusted in the flow direction. The interface also offers, for example, the possibility to use the mouse to graphically identify as ROIs those regions to be analyzed with regard to the flow speed. The measurement results can be shown on this card (e.g. in the form of short movies) directly after the measurement or individual images can be selected by the user and displayed in different enlargements.

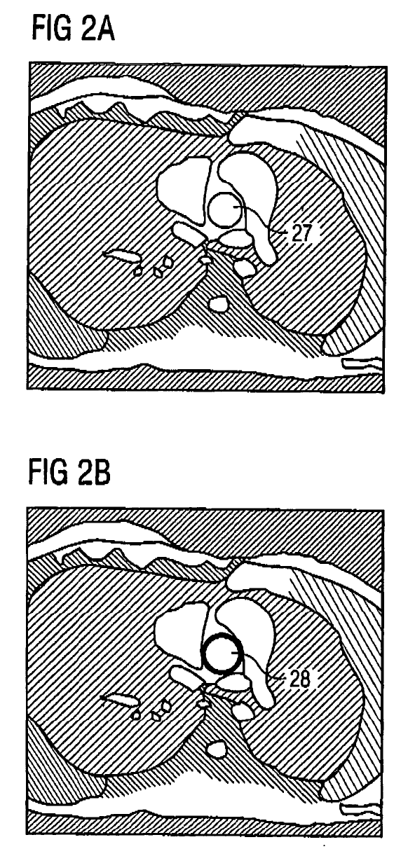

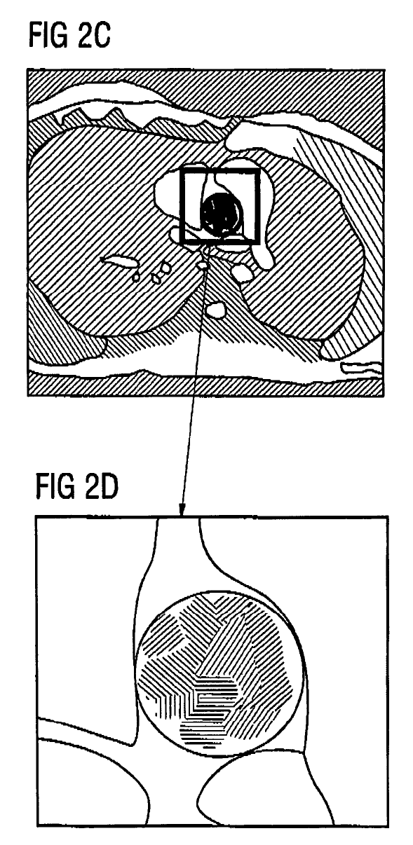

The optimization of the MRT-device for flow measurements and/or the process according to the invention are explained using FIGS. 2A to 2D.

Initially, an overview image (localizer) is acquired of the layer to be measured, in which the vessel regions to be analyzed can be recognized well. In the case of FIG. 2A, the acquired localizer is made transversally (in the through-plane) through the mediastinum. Both sides of the lung can be recognized, in the middle of which the aorta to be measured is located. The speed-coded gradient is generated as a pulse in the flow direction (only for measurement of the speed-coded images), i.e. for through-plane recordings, perpendicularly to the section plane. Likewise, an axial section (in-plane) is possible through the vessel in which fluid is flowing; in this case, the speed-coded gradient must be directed in the section plane in the flow direction.

The planning of the flow direction using the localizer is done such that the user identifies the vessel to be measured as a ROI (manually, for example, with the mouse). In FIG. 2B, the aorta has been marked by a circle. In general, however, several vessel sections can be marked simultaneously in different ways (e.g. rectangle, oval).

Next, the MR-flow measurement is performed such that an ordinary anatomical image and a speed-coded image are alternatingly acquired using a speed-coded gradient. The measurement spans, in case of measurement of the aorta, one or more heartbeat intervals (cardiac cycles), whereby approximately 20 anatomical or speed-coded MRT-images are acquired per heartbeat interval (from systole to systole). During the image acquisition, the ROI is propagated or statically copied by the temporal image series of the speed-coded image series. During the measurement of the image series, a constant adaptation (translation and deformation correction) of the marked ROIs to the irregular contour of the vessel is possible using suitable segmenting algorithms.

From the speed-coded images, the speeds (per pixel or per voxel) are calculated directly after the measurement of the respective image within the respective ROIs. In the process, according to FIG. 2C, the voxels of higher speed are depicted as regions of higher signal intensity.

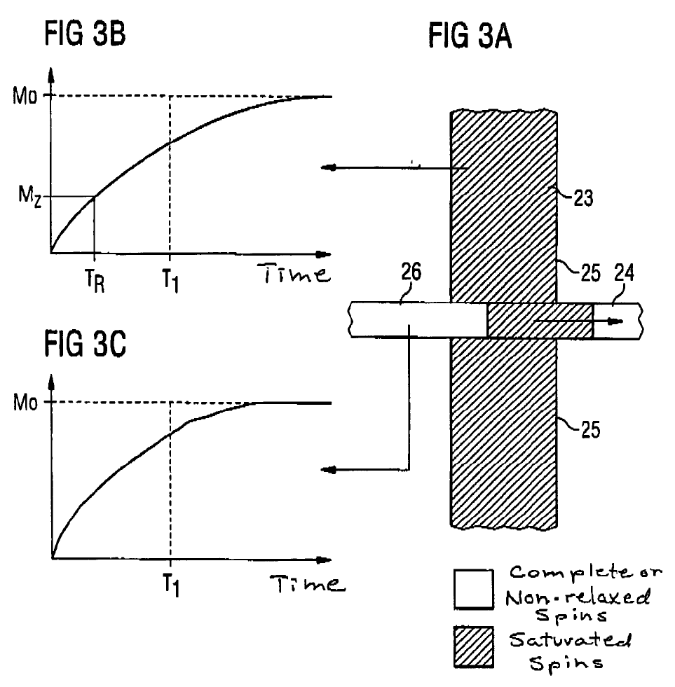

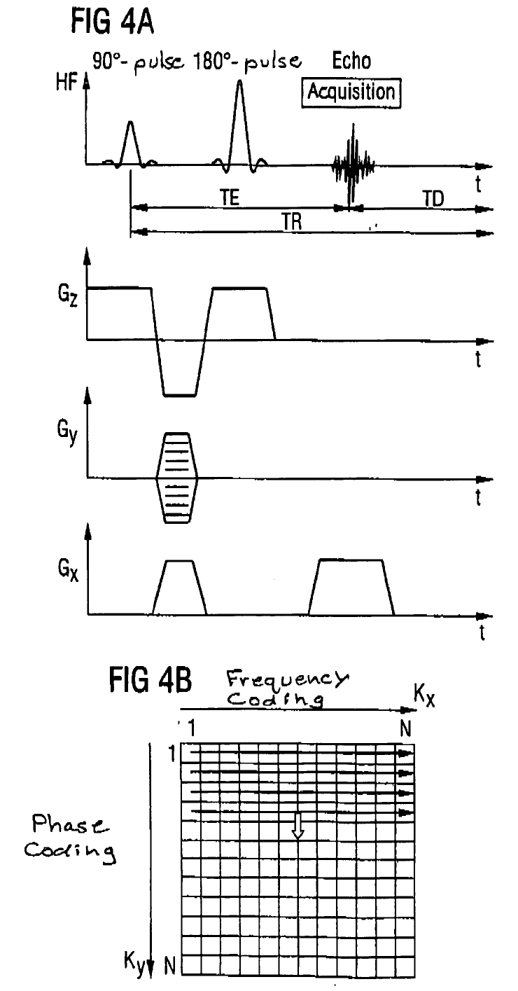

This effect is explained briefly using FIGS. 3A, 3B, and 3C.

As already mentioned, for a magnetic resonance flow measurement, the image slice typically is oriented perpendicularly to the vessels that are to be depicted. In FIG. 3A, an excitation layer 23 of this type is shown schematically. In order to produce an optimal contrast between the stationary tissue and the vessel 24, in which the spins of the stationary tissue 23 are saturated as greatly as possible, the repetition time TR is selected to be as short as possible. When the spins are flipped in rapid succession, there is not enough time for the magnetization to build up again completely in the longitudinal direction. This means that for excitations that follow each other in rapid succession, i.e. during a very brief time period TR, according to FIG. 3B only one small magnetization vector M_{z} is regenerated in the longitudinal direction, which also generates only a few signals after the flipping of the RF-pulse. In this way, the stationary tissue 23 appears very dark in the image. This is called a saturation of the spin.

The spins of the blood 26, which flows through the vessels 23 to be displayed, are only excited if the blood 26 flows into the excitation layer 23. Since prior to entering into the excitation layer 23, the blood still has not experienced any RF-excitation, complete (relaxed) magnetization of the spins of the blood M₀ is available when the blood enters the layer (see FIG. 3C). This has the consequence that blood 26 flowing into the layer, and thus the vascular system through which the blood flows, is shown brighter in the MRT-image than the surrounding stationary tissue 23.

By placing a (phase-) coding gradient in the flow direction, the flowing blood can also be differentiated (coded). The gradient causes an accelerated dephasing (relaxation) of the magnetization; the longer the blood is exposed to the gradient field, the greater the dephasing that occurs and the weaker the magnetic resonance signal. This means that blood flowing quickly exhibits less relaxation and therefore in the later image has a stronger intensity. Between the dephasing that becomes manifested in a defined phase shift φ relative to the magnetization of static material, the speed-coded gradients, the repetition time and the absolute speed of the blood, a mathematical relation exists on the basis of which the speed values of the flowing material can be determined in the ROI.

Both image series—the anatomical series and the speed-coded series—can be shown on the screen as a movie by a temporal sequence of the individual recordings, e.g. at a frequency of 20 images per second. A display of the flow is done according to the invention by, outside of the ROI or (ROIs), the movie of the anatomy that changes because of the heart movement being shown, and within the ROI or (ROIs), the movie of the speed or the flow being shown synchronously. In this way, a flow movie is produced which shows a combination of anatomy and flow information by image superimposition directly after the end of the MRT-measurement (end of scan). The coding of the speed in the ROI is done in a preferred embodiment of the present invention by gray scales or, more user-friendly, by differences in color, as is standard in ultrasound imaging, for example. An image of this type with color coding or gray scale coding is shown in an enlarged section of the ROIs in FIG. 2D.

The presentation according to the invention of the results of flow measurements in the MRT allows the radiologist or the physician to make a diagnosis in a fast and efficient manner. Thus it is possible, for example, to perform a flow measurement directly prior to the heart valves in order to determine, using the color-coded aortas, whether a reflux (e.g. identified by the color green), and thus a leakage of the valves, is present.

In summary, the basic aspects of the process according to the invention and at least some of the advantages resulting from are as follows.

The speed information or the flow information are integrated into the anatomical image. The anatomical image follows according to the movement that is present (cardiac cycle, breathing, etc.), and the speed image is synchronized to the anatomical image. The adaptation of the ROIs to the anatomical movement and thus its display is done using imaging computers during or immediately following the scan. In this way, the user can observe the resultant images individually or in film immediately after the flow measurement and, if necessary, plan additional measurements. The color coding of the flow in ROI makes the diagnosis easier. A loading of the image series into a workstation or into the system computer after the end of the examination and a subsequent post-processing with results, which might make necessary a follow-up examination, is avoided. The process according to the invention optimizes the workflow of an MRT-flow measurement and thus produces a significant time savings both during the measurement and during the evaluation or interpretation of the measurement results (simplified diagnosis). In addition, the patient time in the scanner is minimized.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for speed-resolved flow measurement during a movement cycle in magnetic resonance tomography, comprising the steps of:
acquiring a magnetic resonance tomography overview image of a selected region of a living subject;
displaying the overview image on a screen;
quasi-simultaneously acquiring data for an anatomical image series of the selected region and data for a speed-resolved image series of a region identified within a selected region during the movement cycle, with respective images in said anatomical image series having a time correspondence with respective images in said speed-resolved image series; and
displaying said anatomical image series and said speed-resolved image series on said screen with each image in said speed-resolved image series being integrated in the time-corresponding image of the anatomical image series.

2. A method as claimed in claim 1 comprising segmenting said region identified within said selected region automatically during acquisition of said speed-resolved image series.

3. A method as claimed in claim 1 comprising segmenting said region identified within said selected region immediately after acquisition of said speed-resolved image series.

4. A method as claimed in claim 1 comprising color-coding the images in said speed-resolved image series.

5. A method as claimed in claim 1 comprising displaying said anatomical image series and said speed-resolved image series on said screen immediately after acquiring said data for said anatomical image series and said data for said speed-resolved image series.

6. A method as claimed in claim 5 comprising displaying said anatomical image series and said speed-resolved image series as a movie on said screen.

7. A method as claimed in claim 1 comprising manually identifying, on said screen, said region within said selected region.

8. A method as claimed in claim 1 comprising identifying a plurality of regions within said selected region during the movement cycle, and acquiring data for a speed-resolved image series for each of said regions.

9. A method as claimed in claim 1 comprising acquiring said data for said anatomical image series and said data for said speed-resolved image series for a time, as said movement cycle, selected from the group consisting of a breathing cycle of said subject and an art cycle of said subject.

10. A method as claimed in claim 1 comprising acquiring said data for each of said anatomical image series and said speed-resolved image series at approximately 20 images per movement cycle.

11. A magnetic resonance tomography apparatus comprising:
a magnetic resonance scanner adapted to receive a living subject therein;
a control computer for operating said magnetic resonance scanner;
a display screen connected to said control computer; and
said control computer operating said magnetic resonance scanner for acquiring a magnetic resonance tomography overview image of a selected region of a living subject, displaying the overview image on a screen, quasi-simultaneously acquiring data for an anatomical image series of the selected region and data for a speed-resolved image series of a region identified within a selected region during the movement cycle, with respective images in said anatomical image series having a time correspondence with respective images in said speed-resolved image series, and displaying said anatomical image series and said speed-resolved image series on said screen with each image in said speed-resolved image series being integrated in the time-corresponding image of the anatomical image series.

12. A computer software product loadable into a control computer of a magnetic resonance tomography apparatus, including a magnetic resonance scanner operated by the control computer and a display screen connected to the control computer, said computer program product running in said control computer and programming said control computer to:
acquire a magnetic resonance tomography overview image of a selected region of a living subject,
display the overview image on a screen,
quasi-simultaneously acquire data for an anatomical image series of the selected region and data for a speed-resolved image series of a region identified within a selected region during the movement cycle, with respective images in said anatomical image series having a time correspondence with respective images in said speed-resolved image series, and
display said anatomical image series and said speed-resolved image series on said screen with each image in said speed-resolved image series being integrated in the time-corresponding image of the anatomical image series.
